(19) United States
(12) Patent Application Publication
(10) Pub. No.: US 2026/0102354 A1
(43) Pub. Date: Apr. 16, 2026

(54) PHARMACEUTICAL FORMULATIONS COMPRISING NALTREXONE AND/OR BUPROPION

(71) Applicant: Nalpropion Pharmaceuticals LLC, Brentwood, TN (US)

(72) Inventors: Rob Churchill, Nolensville, TN (US); Aaron Chesnut, Nolensville, TN (US)

(73) Assignee: Nalpropion Pharmaceuticals LLC, Brentwood, TN (US)

(21) Appl. No.: 19/211,696

(22) Filed: May 19, 2025

Related U.S. Application Data
(62) Division of application No. 18/917,080, filed on Oct. 16, 2024, now Pat. No. 12,303,604.

(51) Int. Cl. A61K 9/20; A61K 9/00; A61K 31/137; A61K 31/485

(52) U.S. Cl. CPC A61K 9/2081; A61K 9/0053; A61K 9/2013; A61K 9/2054; A61K 31/137; A61K 31/485

(57) ABSTRACT

Described are pharmaceutical formulations comprising naltrexone and/or bupropion. Such pharmaceutical formulations can comprise extended-release, multilayer beads. Also described are methods of administering such formulations, such as for the treatment of overweight or obesity.

### CROSS-REFERENCE TO RELATED APPLICATIONS

This application is a divisional of application Ser. No. 18/917,080, filed Oct. 16, 2024. The disclosures of all applications cited in this paragraph are incorporated herein in their entirety by reference.

### TECHNICAL FIELD

The present disclosure generally relates to pharmaceutical formulations comprising naltrexone and/or bupropion.

### BACKGROUND

CONTRAVE® (naltrexone hydrochloride and bupropion hydrochloride) extended-release tablets is currently indicated as an adjunct to a reduced-calorie diet and increased physical activity for chronic weight management in adults with an initial body mass index (BMI) of: 30 kg/m² or greater (obese); or 27 kg/m² or greater (overweight) in the presence of at least one weight-related comorbidity (e.g., hypertension, type 2 diabetes mellitus, or dyslipidemia). However, there is a need for new formulations of naltrexone and/or bupropion for the treatment of disorders such as overweight or obesity.

### SUMMARY

One aspect of the present disclosure relates to an extended-release, multilayer particulate (e.g. bead) comprising naltrexone or a salt thereof. In various embodiments of this aspect, the extended-release, multilayer bead comprises: a core particle, wherein the core particle is drug-free; an optional seal coating coated on the surface of the core particle; a drug layer comprising naltrexone, or a salt thereof, coated on the surface of the core particle or, when present, the seal coating; a non-aqueous barrier coating coated on the surface of the drug layer; an extended release coating coated on the surface of the barrier coating; and an optional top coating coated on the surface of the extended release coating.

In one or more embodiments, the naltrexone-containing bead has an average diameter of about 500-900 μm, such as about 600-750 μm.

In one or more embodiments, the naltrexone is present in the drug layer as the hydrochloride salt. In one or more embodiments, the naltrexone comprises an anhydrous polymorph of the hydrochloride salt (Form L). In one or more embodiments, the naltrexone hydrochloride comprises an ethanol solvate (Form F). In one or more embodiments, the naltrexone hydrochloride comprises an anhydrous polymorph (Form L) and/or an ethanol solvate (Form F). In one or more embodiments, the naltrexone hydrochloride comprises greater than 99% Form L and less than 1% Form F. In one or more embodiments, the naltrexone hydrochloride is substantially free of amorphous naltrexone hydrochloride.

In one or more embodiments, the drug layer comprises about 50% to about 95% of naltrexone, or a salt thereof. In one or more embodiments, the extended-release, multilayer bead comprises about 10% to about 80% of naltrexone, or a salt thereof.

In one or more embodiments, the drug-free core particle is selected from a microcrystalline cellulose particle, a silica particle, and a sugar particle. In one or more embodiments, the drug-free core particle is a microcrystalline cellulose particle.

In one or more embodiments, the drug-free core particle is spherical. In one or more embodiments, the drug-free core particle has an average diameter of 200-800 μm, such as 350-500 μm.

In one or more embodiments, the drug layer further comprises a low viscosity binder. In one or more embodiments, the binder is selected from Hypromellose 3 cps, Hypromellose 5 cps, and Hydroxypropyl cellulose. In one or more embodiments, the binder is Hydroxypropyl cellulose Klucel EXF. In one or more embodiments, the ratio of naltrexone, or a salt thereof, to binder is about 100:1 to about 10:1.

In one or more embodiments, the non-aqueous barrier coating comprises a low viscosity binder. In one or more embodiments, the binder is selected from Hypromellose 3 cps, Hypromellose 5 cps, and Hydroxypropyl cellulose. In one or more embodiments, the binder is Hydroxypropyl cellulose Klucel EXF.

In one or more embodiments, the non-aqueous barrier coating is prepared under anhydrous conditions. In one or more embodiments, the non-aqueous barrier coating is prepared using a solvent selected from the group consisting of Class 3 solvents. In one or more embodiments, the solvent is selected from acetic acid, acetone, ethanol and combinations thereof. In one or more embodiments, the solvent comprises dehydrated ethanol.

In one or more embodiments, the extended-release coating achieves a mean peak naltrexone concentration (Cₘₐₓ) of about 1.4 ng/ml, a time to peak concentration (Tₘₐₓ) of about 2 hours, and/or an extent of exposure (AUC_{0-inf}) of about 8.4 ng·hr/mL after administration of a plurality of the extended-release multilayer beads having a total naltrexone loading of about 16 mg.

In one or more embodiments, the extended-release coating comprises a release controlling polymer and a hydrophilic plasticizer. In one or more embodiments, the release controlling polymer comprises ethyl cellulose. In one or more embodiments, the release controlling polymer is present in an amount of about 5% to about 25% by weight of the extended-release, multilayer bead.

In one or more embodiments, the hydrophilic plasticizer comprises triethyl citrate. In one or more embodiments, the hydrophilic plasticizer is present in an amount of about 0.5% to 5% by weight of the extended-release, multilayer bead. In one or more embodiments, the hydrophilic plasticizer is present in an amount of about 5% to about 15% of dry polymer concentration.

In one or more embodiments, the extended-release coating comprises a pore former. In one or more embodiments, the pore former comprises Hypromellose.

In one or more embodiments, the naltrexone-containing bead maintains naltrexone stability for at least 6, 12, 18 or 24 months at room temperature. In one or more embodiments, the naltrexone-containing bead maintains naltrexone stability under accelerated aging conditions of 40° C./75% relative humidity (RH) for 7, 14 or 28 days or 1, 2, 3, 4, 5, 6 or more months. In one or more embodiments, the naltrexone stability comprises one or more of release stability, impurity/degradant stability and/or polymorphic stability.

In one or more embodiments, the naltrexone-containing bead comprises no more than 0.5% 2-chloro-10α-hydroxynaltrexone, based on the weight of naltrexone.

In one or more embodiments, the naltrexone-containing bead comprises no more than 2% impurities, based on the weight of naltrexone.

Another aspect of the present disclosure relates to extended-release, multilayer particulate (e.g. bead) comprising bupropion or a salt thereof. In various embodiments of this aspect, the extended-release, multilayer bead comprises: a core particle comprising bupropion, or a salt thereof; an optional seal coating coated on the surface of the core particle; an extended-release coating coated on the surface of the core particle, or if present, on the surface of the seal coating; and an optional top coating coated on the surface of the extended-release coating.

In one or more embodiments, the bupropion is present as the hydrochloride salt. In one or more embodiments, the bupropion, or a salt thereof, is micronized (D10=1 μm, D50-6 μm and D90=40 μm). In one or more embodiments, the bupropion, or a salt thereof, has a bulk density of about 0.1 to 0.4 g/cc.

In one or more embodiments, the bupropion-containing core particles comprise ≥70% w/w bupropion, or a salt thereof. In one or more embodiments, the bupropion-containing beads comprise 60 to 80% w/w bupropion, or a salt thereof.

In one or more embodiments, the bupropion-containing core particle is prepared using a pelletization process. In one or more embodiments, the bupropion-containing core particle is prepared using extrusion. In one or more embodiments, the bupropion-containing core particle is prepared using extrusion-spheronization.

In one or more embodiments, the average diameter of the bupropion-containing core particle is about 1000-1500 μm, such as about 1200 μm.

In one or more embodiments, the bupropion-containing bead comprises no more than 3.2% impurities, based on the weight of bupropion. In one or more embodiments, the bupropion-containing bead comprises no more than 1% (2R,3R,5R)-2-(3-chlorophenyl)-2-hydroxy-3-methylthiomorpholine-5-carboxylic acid (RRR-CHMTCA), based on the weight of bupropion. In one or more embodiments, the bupropion-containing bead comprises no more than 0.5% (2S,3S,5R)-2-(3-chlorophenyl)-2-hydroxy-3-methylthiomorpholine-5-carboxylic acid (SSR-CHMTCA), based on the weight of bupropion.

Another aspect of the disclosure relates to an oral dosage form comprising a plurality of the extended-release, multilayer multiparticulates (e.g. beads) comprising naltrexone or salt thereof.

Another aspect of the disclosure relates to an oral dosage form comprising a plurality of the extended-release, multilayer multiparticulates (e.g. beads) comprising bupropion or salt thereof.

Another aspect of the disclosure relates to oral dosage form comprising a plurality of the extended-release, multilayer multiparticulates (e.g. beads) comprising naltrexone or salt thereof and a plurality of the extended-release, multilayer multiparticulates (e.g. beads) comprising bupropion or salt thereof.

In various aspects, the dosage form has an in vitro bupropion dissolution profile in a dissolution test of USP Apparatus 1 Basket Method at 100 rpm in a dissolution medium of water at 37° C. of: less than 30% of the bupropion released in one hour; and/or less than 60% of the bupropion released in two hours.

In various aspects, the dosage form has an in vitro naltrexone dissolution profile in a dissolution test of USP Apparatus 1 Basket Method at 100 rpm in a dissolution medium of water at 37° C. of: less than 30% of the naltrexone released in one hour; and/or less than 60% of the naltrexone released in two hours.

Another aspect of the present disclosure relates to an oral dosage form comprising a naltrexone extended-release formulation comprising about 8 to about 32 mg of naltrexone or a salt thereof; and a bupropion extended-release formulation comprising about 90 mg to about 360 mg of bupropion or a salt thereof. In various embodiments of this aspect, the bupropion extended-release formulation has an in vitro bupropion dissolution profile in a dissolution test of USP Apparatus 1 Basket Method at 100 rpm in a dissolution medium of water at 37° C. of: less than 30% of the bupropion released in one hour; and/or less than 60% of the bupropion released in two hours. In various embodiments of this aspect, the naltrexone extended-release formulation has an in vitro naltrexone dissolution profile in a dissolution test of USP Apparatus 1 Basket Method at 100 rpm in a dissolution medium of water at 37° C. of: less than 30% of the naltrexone released in one hour; and/or less than 60% of the naltrexone released in two hours.

In one or more embodiments, the in vitro naltrexone dissolution profile in the dissolution test is: less than 20% of the naltrexone released in one hour; and/or less than 50% of the naltrexone released in two hours. In one or more embodiments, the in vitro naltrexone dissolution profile in the dissolution test is: 2% to 20% of the naltrexone released in one hour; and/or 10 to 50% of the naltrexone released in two hours; and/or 60% to 90% of the naltrexone released in four hours. In one or more embodiments, the in vitro naltrexone dissolution profile in the dissolution test is: 2% to 10% of the naltrexone released in one hour; and/or 20 to 40% of the naltrexone released in two hours.

In one or more embodiments, the in vitro bupropion dissolution profile in the dissolution test is: less than 20% of the bupropion released in one hour; and/or less than 50% of the bupropion released in two hours. In one or more embodiments, the in vitro bupropion dissolution profile in the dissolution test is: 2% to 20% of the bupropion released in one hour; and/or 10 to 50% of the bupropion released in two hours; and/or 50% to 90% of the bupropion released in four hours. In one or more embodiments, the in vitro bupropion dissolution profile in the dissolution test is: 2% to 10% of the bupropion released in one hour; and/or 20 to 40% of the bupropion released in two hours.

In one or more embodiments, the oral dosage form comprises about 8 mg of naltrexone or salt thereof and about 90 mg of bupropion or salt thereof.

In one or more embodiments, the oral dosage form comprises about 16 mg of naltrexone or salt thereof and about 180 mg of bupropion or salt thereof.

In one or more embodiments, the oral dosage form comprises about 24 mg of naltrexone or salt thereof and about 270 mg of bupropion or salt thereof.

In one or more embodiments, the oral dosage form comprises about 32 mg of naltrexone or salt thereof and about 360 mg of bupropion or salt thereof.

In one or more embodiments, administration of the oral dosage form comprising 32 mg of naltrexone to a group of subjects at steady state provides an average maximum naltrexone plasma concentration Cₘₐₓ of 0.8 to 2.3 ng/mL, such as 1.1 to 1.8 ng/mL.

In one or more embodiments, administration of the oral dosage form comprising 32 mg of naltrexone to a group of subjects at steady state provides an average naltrexone plasma area under the curve AUC₀₋₂₄ₕ of 10 to 28 ng*h/mL, such as 13 to 22 ng*h/mL.

In one or more embodiments, administration of the oral dosage form comprising 360 mg of bupropion to a group of subjects at steady state provides an average maximum bupropion plasma concentration Cₘₐₓ of 120 to 330 ng/mL, such as 150 to 260 ng/mL.

In one or more embodiments, administration of the oral dosage form comprising 360 mg of bupropion to a group of subjects at steady state provides an average bupropion plasma area under the curve AUC₀₋₂₄ₕ of 1,700 to 4,500 ng*h/mL, such as 2,200 to 3,600 ng*h/mL.

In one or more embodiments, the naltrexone salt is naltrexone hydrochloride. In one or more embodiments, the naltrexone comprises an anhydrous polymorph of the hydrochloride salt (Form L). In one or more embodiments, the naltrexone hydrochloride comprises an ethanol solvate (Form F). In one or more embodiments, the naltrexone hydrochloride comprises an anhydrous polymorph (Form L) and/or an ethanol solvate (Form F). In one or more embodiments, the naltrexone hydrochloride comprises greater than 99% Form L and less than 1% Form F. In one or more embodiments, the naltrexone hydrochloride is substantially free of amorphous naltrexone hydrochloride.

In one or more embodiments, the oral dosage form, when administered to a human subject once daily, is bioequivalent to a US FDA-approved trilayer tablet dosage form comprising naltrexone hydrochloride and bupropion hydrochloride (e.g. CONTRAVE®) administered to a human subject twice daily, under the bioequivalence parameters of: (a) a 90% Confidence Interval for AUC which is between 80% and 125%, and (b) a 90% Confidence Interval for Cₘₐₓ, which is between 80% and 125%.

Another aspect of the present disclosure relates to an oral dosage form that, when administered to a human subject once daily, is bioequivalent to a US FDA-approved trilayer tablet dosage form comprising naltrexone hydrochloride and bupropion hydrochloride (e.g. CONTRAVE®) administered to a human subject twice daily, under the bioequivalence parameters of: (a) a 90% Confidence Interval for AUC which is between 80% and 125%, and (b) a 90% Confidence Interval for Cₘₐₓ, which is between 80% and 125%.

In various embodiments of this aspect, the oral dosage form comprises about 8 mg to about 32 mg of naltrexone, or a salt thereof.

In one or more embodiments, the oral dosage form comprises about 90 mg to about 360 mg of bupropion, or a salt thereof.

In one or more embodiments, the oral dosage form comprises a capsule.

Another aspect of the present disclosure relates to a method of administering naltrexone and/or bupropion by administering an oral dosage form as described herein.

Another aspect of the present disclosure relates to a method of treating overweight or obesity by administering an oral dosage form as described herein.

In one or more embodiments, the method comprises administering a first oral dosage form comprising about 8 mg of naltrexone or salt thereof and about 90 mg of bupropion or salt thereof, wherein the first oral dosage form is administered once a day for a first week; administering a second oral dosage form comprising about 16 mg of naltrexone or salt thereof and about 180 mg of bupropion or salt thereof, wherein the second oral dosage form is administered once a day for a second week; administering a third oral dosage form comprising about 24 mg of naltrexone or salt thereof and about 270 mg of bupropion or salt thereof, wherein the third oral dosage form is administered once a day for a third week; and administering a fourth oral dosage form comprising about 32 mg of naltrexone or salt thereof and about 360 mg of bupropion or salt thereof, wherein the fourth oral dosage form is administered once a day for a fourth and subsequent weeks.

### BRIEF DESCRIPTION OF THE DRAWINGS

FIG. 1 shows the in vitro dissolution profiles of various naltrexone extended-release formulations;

FIG. 2 shows the in vitro dissolution profiles of various naltrexone extended-release formulations;

FIG. 3 shows the in vitro dissolution profiles of various naltrexone extended-release formulations;

FIG. 4 shows pharmacokinetic simulations of various naltrexone extended-release formulations;

FIG. 5 shows the in vitro dissolution profiles of various bupropion extended-release formulations;

FIG. 6 shows the in vitro dissolution profiles of various bupropion extended-release formulations;

FIG. 7 shows the in vitro dissolution profiles of various bupropion extended-release formulations;

FIG. 8 shows the in vitro dissolution profiles of various bupropion extended-release formulations;

FIG. 9 shows the in vitro dissolution profiles of various bupropion extended-release formulations; and

FIG. 10 shows pharmacokinetic simulations of various bupropion extended-release formulations.

### DETAILED DESCRIPTION

Various aspects of the present disclosure relate to methods and pharmaceutical formulations comprising naltrexone and/or bupropion. In one or more embodiments, the methods and formulations described herein overcome challenges that are unique to naltrexone, bupropion and/or the combination of naltrexone and bupropion. For example, it has been found that (1) naltrexone and bupropion should be physically separated to prevent potential interaction between the two active ingredients; (2) naltrexone hydrochloride is highly hygroscopic and readily converts between different polymorphs/solvates; (3) amorphous naltrexone hydrochloride is not chemically stable and subject to rapid degradation; (4) bupropion dosages commonly prescribed in combination with naltrexone (e.g. 360 mg/day) are relatively high loadings of drug to incorporate into extended-release formulations; and (5) bupropion dosages commonly prescribed in combination with naltrexone (e.g. 360 mg/day) are more than ten times the corresponding naltrexone dosages (e.g. 32 mg/day).

CONTRAVE® (naltrexone hydrochloride and bupropion hydrochloride) extended-release tablets is currently indicated as an adjunct to a reduced-calorie diet and increased physical activity for chronic weight management in adults with an initial body mass index (BMI) of: 30 kg/m² or greater (obese); or 27 kg/m² or greater (overweight) in the presence of at least one weight-related comorbidity (e.g., hypertension, type 2 diabetes mellitus, or dyslipidemia). In various embodiments, the pharmaceutical formulations described herein can also be used for the treatment of overweight or obesity. The prescribing information for CONTRAVE® is hereby incorporated by reference in its entirety.

As used herein, the phrase “US FDA-approved trilayer tablet dosage form comprising naltrexone hydrochloride and bupropion hydrochloride” refers to the trilayer tablet dosage form of CONTRAVE® (naltrexone hydrochloride and bupropion hydrochloride) that was first approved by the US Food and Drug Administration on Sep. 10, 2014, and is currently available as of Oct. 15, 2024 in the US by prescription.

Exemplary doses, dosage forms, formulations and methods of administering naltrexone and bupropion are described in the following patents and patent applications, which are hereby incorporated by reference in their entireties: U.S. Pat. Nos. 7,375,111; 8,916,195; 8,088,786; 8,722,085; 8,815,889; 9,248,123; 9,633,575; 8,969,371; 10,231,962; 11,324,741; U.S. Pat. App. Pub. No. 2011/0028505; U.S. Pat. App. Pub. No. 2013/0252995 and U.S. Pat. App. Pub. No. 2013/0245056.

In some embodiments, the naltrexone and/or bupropion (or salt(s) thereof) is administered once per day. In one or more embodiments, naltrexone and bupropion (or salt(s) thereof) are administered in a single oral dosage form once per day.

In some embodiments, the daily dose of naltrexone can range from about 8 mg to about 32 mg. In some embodiments, the daily dose is about 8 mg, about 16 mg, about 24 mg, or about 32 mg of naltrexone, or a range defined by any two of the preceding values. In one or more embodiments, the naltrexone or pharmaceutically acceptable salt thereof is administered at a dose of about 8 mg per day. In one or more embodiments, the naltrexone or pharmaceutically acceptable salt thereof is administered at a dose of about 16 mg per day. In one or more embodiments, the naltrexone or pharmaceutically acceptable salt thereof is administered at a dose of about 24 mg per day. In one or more embodiments, the naltrexone or pharmaceutically acceptable salt thereof is administered at a dose of about 32 mg per day.

In some embodiments, the daily dose of bupropion can range from about 90 mg to about 360 mg. In some embodiments, the daily dose is about 90 mg, about 180 mg, about 270 mg, or about 360 mg of bupropion, or a range defined by any two of the preceding values. In one or more embodiments, the bupropion or pharmaceutically acceptable salt thereof is administered at a dose of about 90 mg per day. In one or more embodiments, the bupropion or pharmaceutically acceptable salt thereof is administered at a dose of about 180 mg per day. In one or more embodiments, the bupropion or pharmaceutically acceptable salt thereof is administered at a dose of about 270 mg per day. In one or more embodiments, the bupropion or pharmaceutically acceptable salt thereof is administered at a dose of about 360 mg per day.

The compositions described herein may be distributed, provided to a patient for self-administration, or administered to an individual.

In some embodiments, that naltrexone and/or bupropion is provided or administered as an oral dosage form. In some embodiments, the oral dosage form is in the form of a pill, tablet, core, capsule, caplet, loose powder, solution, or suspension. In a preferred embodiment, the oral dosage form is in the form of a pill, tablet, or capsule. In some embodiments, the combined naltrexone/bupropion therapy is provided in a single oral dosage form. In some embodiments, the combined naltrexone/bupropion therapy is provided in a capsule comprising multilayer, extended-release multiparticulates (e.g. beads). As used herein, the term “bead” refers to a particulate comprising the listed components. The bead does not necessarily need to be spherical, but can be any regular or irregular shaped particle. In some embodiments, the bead has a substantially ellipsoid shape. In some embodiments, the bead is substantially spherical.

In various embodiments, the naltrexone or salt thereof is provided in multilayer, extended-release beads. In one or more embodiments, the extended-release, multilayer bead comprises: a core particle, wherein the core particle is drug-free; an optional seal coating coated on the surface of the core particle; a drug layer comprising naltrexone, or a salt thereof, coated on the surface of the core particle or, when present, the seal coating; a non-aqueous barrier coating coated on the surface of the drug layer; an extended release coating coated on the surface of the barrier coating; and an optional top coating coated on the surface of the extended release coating.

In one or more embodiments, the drug layer comprises about 50% to about 95% of naltrexone, or a salt thereof. In various embodiments, the drug layer comprises at least 50%, 55%, 60%, 65%, 70%, 75% or 80% by weight naltrexone, or salt thereof. In various embodiments, the drug layer comprises less than 99%, 98%, 97%, 96%, 95%, 94%, 93%, 92%, 91%, 90% or 85% by weight naltrexone, or salt thereof.

In one or more embodiments, the extended-release, multilayer bead comprises about 10% to about 80% of naltrexone, or a salt thereof. In various embodiments, the extended-release, multilayer bead comprises at least 10%, 15%, 20%, 25 or 30% by weight naltrexone, or salt thereof. In various embodiments, the extended-release, multilayer bead comprises less than 50%, 45%, 40%, 35%, 30% or 25% by weight naltrexone, or salt thereof.

In one or more embodiments, the drug-free core particle for the naltrexone-containing bead is selected from a microcrystalline cellulose particle, a silica particle, and a sugar particle. In one or more embodiments, the drug-free core particle is a microcrystalline cellulose particle.

In one or more embodiments, the drug-free core particle for the naltrexone-containing bead is spherical. In one or more embodiments, the drug-free core particle has an average diameter of 200-800 μm, such as 350-500 μm.

In one or more embodiments, the drug layer for the naltrexone-containing bead further comprises a low viscosity binder. In one or more embodiments, the binder is selected from Hypromellose 3 cps, Hypromellose 5 cps, and Hydroxypropyl cellulose. In one or more embodiments, the binder is Hydroxypropyl cellulose Klucel EXF. In one or more embodiments, the weight ratio of naltrexone, or a salt thereof, to binder is about 100:1 to about 10:1, such as about 100:1, about 90:1, about 80:1, about 70:1, about 60:1, about 50:1, about 40:1, about 30:1, about 25:1, about 20:1, about 15:1 or about 10:10.

In one or more embodiments, the drug layer comprising naltrexone is applied to the drug-free core particle under anhydrous conditions. In one or more embodiments, the drug layer is prepared using a solvent selected from the group consisting of Class 3 solvents. In one or more embodiments, the solvent is selected from acetic acid, acetone, ethanol and combinations thereof. In one or more embodiments, the solvent comprises dehydrated ethanol.

In one or more embodiments, the non-aqueous barrier coating comprises a low viscosity binder. In one or more embodiments, the binder is selected from Hypromellose 3 cps, Hypromellose 5 cps, and Hydroxypropyl cellulose. In one or more embodiments, the binder is Hydroxypropyl cellulose Klucel EXF.

In one or more embodiments, the non-aqueous barrier coating is prepared under anhydrous conditions. In one or more embodiments, the non-aqueous barrier coating is prepared using a solvent selected from the group consisting of Class 3 solvents. In one or more embodiments, the solvent is selected from acetic acid, acetone, ethanol and combinations thereof. In one or more embodiments, the solvent comprises dehydrated ethanol.

In one or more embodiments, the extended-release coating for the naltrexone-containing bead achieves a mean peak naltrexone concentration (Cₘₐₓ) of about 1.4 ng/ml, a time to peak concentration (Tₘₐₓ) of about 2 hours, and/or an extent of exposure (AUC_{0-inf}) of about 8.4 ng·hr/mL after administration of a plurality of the extended-release multilayer beads having a total naltrexone loading of about 16 mg.

In one or more embodiments, the extended-release coating comprises a release controlling polymer and a hydrophilic plasticizer. In one or more embodiments, the release controlling polymer comprises ethyl cellulose. In one or more embodiments, the wherein the release controlling polymer is present in an amount of about 5% to about 25% by weight of the extended-release, multilayer bead.

In one or more embodiments, the hydrophilic plasticizer comprises triethyl citrate. In one or more embodiments, the hydrophilic plasticizer is present in an amount of about 0.5% to 5% by weight of the extended-release, multilayer bead. In one or more embodiments, the hydrophilic plasticizer is present in an amount of about 5% to about 15% of dry polymer concentration.

In one or more embodiments, the extended-release coating comprises a pore former. In one or more embodiments, the pore former comprises Hypromellose. In one or more embodiments, the pore former is present in an amount of about 10% to about 40% of the release controlling polymer, such as about 10%, about 15%, about 20%, about 25%, about 30%, about 35% or about 40%.

In one or more embodiments, the naltrexone-containing bead maintains naltrexone stability for at least 6, 12, 18 or 24 months at room temperature. In one or more embodiments, the naltrexone-containing bead maintains naltrexone stability under accelerated aging conditions of 40° C./75% relative humidity (RH) for 7, 14 or 28 days or 1, 2, 3, 4, 5, 6 or more months. In one or more embodiments, the naltrexone stability comprises one or more of release stability, impurity/degradant stability and/or polymorphic stability.

In one or more embodiments, the naltrexone-containing bead comprises no more than 2% impurities, based on the weight of naltrexone. In one or more embodiments, the naltrexone-containing bead comprises no more than 0.5% 2-chloro-10α-hydroxynaltrexone, based on the weight of naltrexone.

In one or more embodiments, the naltrexone salt comprises naltrexone hydrochloride. In one or more embodiments, the naltrexone hydrochloride comprises an anhydrous polymorph of the hydrochloride salt (Form L). In one or more embodiments, the naltrexone hydrochloride comprises an ethanol solvate of the hydrochloride salt (Form F). In one or more embodiments, the naltrexone hydrochloride comprises an anhydrous polymorph (Form L) and/or an ethanol solvate (Form F). In one or more embodiments, the naltrexone hydrochloride comprises greater than 90%, 95%, 99%, or 99.5% Form L and less than 10%, 5%, 1% or 0.5% Form F. In one or more embodiments, the naltrexone hydrochloride has an X-Ray powder diffractogram (XRPD) that is consistent with Form L. In one or more embodiments, the naltrexone hydrochloride has an XRPD that is consistent with Form F. In one or more embodiments, the naltrexone hydrochloride has an XRPD that is consistent with Forms F and L. In one or more embodiments, the naltrexone hydrochloride is substantially free of amorphous naltrexone hydrochloride.

In various embodiments, the bupropion or salt thereof is provided in multilayer, extended-release beads. In one or more embodiments, the extended-release, multilayer bead comprises: a core particle comprising bupropion, or a salt thereof; an optional seal coating coated on the surface of the core particle; an extended-release coating coated on the surface of the core particle, or if present, on the surface of the seal coating; and an optional top coating coated on the surface of the extended-release coating.

In one or more embodiments, the bupropion is present as the hydrochloride salt. In one or more embodiments, the bupropion, or a salt thereof, is micronized (D10=1 μm, D50=6 μm and D90=40 μm). In one or more embodiments, the bupropion, or a salt thereof, has a bulk density of about 0.1 to 0.4 g/cc.

In one or more embodiments, the bupropion-containing core particles comprise ≥70% w/w bupropion, or a salt thereof. In one or more embodiments, the core particles comprise at least 60%, 65%, 70%, 75%, 80% or 85% by weight bupropion, or a salt thereof. In one or more embodiments, the core particles comprise less than 95%, 90%, 85% or 80% by weight bupropion, or a salt thereof.

In one or more embodiments, the bupropion-containing beads comprise 60 to 80% w/w bupropion, or a salt thereof. In one or more embodiments, the core particles comprise at least 40%, 45%, 50%, 55%, 60% or 65% by weight bupropion, or a salt thereof. In one or more embodiments, the core particles comprise less than 95%, 90%, 85%, 80%, 75% or 70% by weight bupropion, or a salt thereof.

In one or more embodiments, the bupropion-containing core particle is prepared using a pelletization process. In one or more embodiments, the bupropion-containing core particle is prepared using extrusion. In one or more embodiments, the bupropion-containing core particle is prepared using extrusion-spheronization.

In one or more embodiments, the average diameter of the bupropion-containing core particle is about 1000-1500 μm, such as about 1200 μm.

In one or more embodiments, the bupropion-containing bead comprises no more than 3.2% impurities, based on the weight of bupropion. In one or more embodiments, the bupropion-containing bead comprises no more than 1% (2R,3R,5R)-2-(3-chlorophenyl)-2-hydroxy-3-methylthiomorpholine-5-carboxylic acid (RRR-CHMTCA), based on the weight of bupropion. In one or more embodiments, the bupropion-containing bead comprises no more than 0.5% (2S,3S,5R)-2-(3-chlorophenyl)-2-hydroxy-3-methylthiomorpholine-5-carboxylic acid (SSR-CHMTCA), based on the weight of bupropion.

In one or more embodiments, the bupropion salt comprises bupropion hydrochloride. In one or more embodiments, the bupropion, or a salt thereof, is micronized (D10=1 μm, D50=6 μm and D90=40 μm).

In one or more embodiments, the bupropion, or a salt thereof, has a bulk density of about 0.1 to 0.4 g/cc. In various embodiments, the bulk density is about 0.1, 0.15, 0.2, 0.25, 0.3, 0.35, 0.4 g/cc or a range defined by any two of the preceding values.

In some embodiments, at least one of naltrexone and bupropion is administered with varying amounts during treatment. In some embodiments, the dose of naltrexone and bupropion is administered in an escalating manner. In one or more embodiments, 8 mg of naltrexone (or salt thereof) and 90 mg of bupropion (or salt thereof) are administered daily for a first week. In one or more embodiments, 16 mg of naltrexone (or salt thereof) and 180 mg of bupropion (or salt thereof) are administered daily for a second week. In one or more embodiments, 24 mg of naltrexone (or salt thereof) and 270 mg of bupropion (or salt thereof) are administered daily for a third week. In one or more embodiments, 32 mg of naltrexone (or salt thereof) and 360 mg of bupropion (or salt thereof) are administered daily thereafter.

In some embodiments, at least one of naltrexone or bupropion is in an extended-release formulation. Although specific reference is made to extended-release, multilayer bead formulations, other formulations may be utilized to obtain the dissolution profiles as described herein.

In some embodiments, the naltrexone is in an extended-release formulation. In various embodiments, the naltrexone extended-release formulation has an in vitro naltrexone dissolution profile in a dissolution test of USP Apparatus 1 Basket Method at 100 rpm in a dissolution medium of water at 37° C. of: less than 30% of the naltrexone released in one hour; and/or less than 60% of the naltrexone released in two hours.

In various embodiments, the in vitro naltrexone dissolution profile provides less than 30%, 25%, 20%, 15% or 10% of naltrexone released in one hour. In various embodiments, the in vitro naltrexone dissolution profile provides at least 1%, 2%, 3%, 4%, 5%, 6%, 7%, 8%, 9% or 10% of naltrexone released in one hour.

In various embodiments, the in vitro naltrexone dissolution profile provides less than 60%, 55%, 50%, 45% or 40% of naltrexone released in two hours. In various embodiments, the in vitro naltrexone dissolution profile provides at least 10%, 15%, 20%, 25%, 30%, 35% or 40% of naltrexone released in two hours.

In various embodiments, the in vitro naltrexone dissolution profile provides less than 80%, 75%, 70%, 65% or 60% of naltrexone released in three hours. In various embodiments, the in vitro naltrexone dissolution profile provides at least 30%, 35%, 40%, 45%, 50%, 55% or 60% of naltrexone released in three hours.

In various embodiments, the in vitro naltrexone dissolution profile provides less than 95%, 90%, 85% or 80% of naltrexone released in four hours. In various embodiments, the in vitro naltrexone dissolution profile provides at least 50%, 55%, 60%, 65%, 70%, 75% or 80% of naltrexone released in four hours.

In various embodiments, the in vitro naltrexone dissolution profile provides less than 99%, 98%, 97%, 96%, 95%, 94%, 93%, 92%, 91% or 90% of naltrexone released in five hours. In various embodiments, the in vitro naltrexone dissolution profile provides at least 60%, 65%, 70%, 75%, 80%, 85% or 90% of naltrexone released in five hours.

In various embodiments, the in vitro naltrexone dissolution profile provides less than 99%, 98%, 97%, 96%, 95%, 94%, 93%, 92%, 91% or 90% of naltrexone released in six hours. In various embodiments, the in vitro naltrexone dissolution profile provides at least 80%, 85%, 90%, 91%, 92%, 93%, 94%, 95%, 96% or 97% of naltrexone released in six hours.

In some embodiments, the bupropion is in an extended-release formulation. In various embodiments, the bupropion extended-release formulation has an in vitro bupropion dissolution profile in a dissolution test of USP Apparatus 1 Basket Method at 100 rpm in a dissolution medium of water at 37° C. of: less than 30% of the bupropion released in one hour; and/or less than 60% of the bupropion released in two hours.

In various embodiments, the in vitro bupropion dissolution profile provides less than 30%, 25%, 20%, 15% or 10% of bupropion released in one hour. In various embodiments, the in vitro bupropion dissolution profile provides at least 1%, 2%, 3%, 4%, 5%, 6%, 7%, 8%, 9%, 10%, 11%, 12%, 13%, 14% or 15% of bupropion released in one hour.

In various embodiments, the in vitro bupropion dissolution profile provides less than 60%, 55%, 50%, 45%, 40% or 35% of bupropion released in two hours. In various embodiments, the in vitro bupropion dissolution profile provides at least 10%, 15%, 20%, 25%, 30%, 35% or 40% of bupropion released in two hours.

In various embodiments, the in vitro bupropion dissolution profile provides less than 80%, 75%, 70%, 65%, 60%, 55% or 50% of bupropion released in three hours. In various embodiments, the in vitro bupropion dissolution profile provides at least 20%, 25%, 30%, 35%, 40%, 45%, 50% or 55% of bupropion released in three hours.

In various embodiments, the in vitro bupropion dissolution profile provides less than 95%, 90%, 85%, 80%, 75%, 70%, 65% or 60% of bupropion released in four hours. In various embodiments, the in vitro bupropion dissolution profile provides at least 40%, 45%, 50%, 55%, 60%, 65% or 70% of bupropion released in four hours.

In various embodiments, the in vitro bupropion dissolution profile provides less than 99%, 98%, 97%, 96%, 95%, 94%, 93%, 92%, 91%, 90%, 85%, 80%, 75% or 70% of bupropion released in five hours. In various embodiments, the in vitro bupropion dissolution profile provides at least 50%, 55%, 60%, 65%, 70%, 75%, 80%, 85% or 90% of bupropion released in five hours.

In various embodiments, the in vitro bupropion dissolution profile provides less than 99%, 98%, 97%, 96%, 95%, 94%, 93%, 92%, 91%, 90%, 85%, 80% or 75% of bupropion released in six hours. In various embodiments, the in vitro bupropion dissolution profile provides at least 60%, 65%, 70%, 75%, 80%, 85%, 90%, 91%, 92%, 93%, 94%, 95%, 96% or 97% of bupropion released in six hours.

In various embodiments, the release profiles of naltrexone and/or bupropion are achieved through the use of an extended-release coating. In one or more embodiments, the extended-release coating comprises a release controlling polymer. In one or more embodiments, the extended-release coating further comprises a pore former. Incorporation of higher amounts of pore former in the extended-release coating led to faster release rates. Utilization of thicker extended-release coatings (higher polymer weight gain) for the beads lead to slower release rates. Thus, in one or mor embodiments, the release rate of naltrexone and/or bupropion can be varied by incorporating varying thickness/polymer weight gain and/or varying pore former content.

In some embodiments, naltrexone and bupropion are administered individually. In some embodiments, naltrexone and bupropion are administered in a single pharmaceutical composition comprising naltrexone and bupropion. In some embodiments, at least one of naltrexone or bupropion is administered with a physiologically acceptable carrier, diluent, or excipient, or a combination thereof. Reference herein to the use or administration of naltrexone and naltrexone/bupropion combinations is understood to include all modes of administration disclosed or referred to herein, including without limitation separate administration, administration in a single dosage form, administration in the form of salts, and/or metabolites, and/or administration in sustained release forms. Techniques for formulation and administration of the compounds of the instant application may be found in “Remington's Pharmaceutical Sciences,” Mack Publishing Co., Easton, Pa., 18th edition, 1990, which is incorporated herein by reference in its entirety.

In some embodiments, naltrexone is administered prior to bupropion. In some embodiments, naltrexone is administered subsequent to bupropion. In some embodiments, naltrexone and bupropion are co-administered. As used herein, co-administration includes administration in a single dosage form, or separate dosage forms that are administered at, or nearly at, the same time.

In one or more embodiments, administration of the oral dosage form comprising 16 mg of naltrexone provides a mean peak naltrexone concentration (Cₘₐₓ) of about 1.4 ng/ml, a time to peak concentration (Tₘₐₓ) of about 2 hours, and/or an extent of exposure (AUC_{0-inf}) of about 8.4 ng·hr/mL.

In one or more embodiments, the oral dosage form described herein the administered to a group of subjects. In various embodiments, the group of subjects is at least 10, 15, 20, 25, 30, 40, 50, 60, 70, 80, 90 or 100 or more subjects. In various embodiments, the oral dosage form is repeatedly administered to the patients until the subjects reach steady state, which is when the drug absorption and drug elimination between successive administrations are approximately equal.

In one or more embodiments, administration of the oral dosage form comprising 32 mg of naltrexone to a group of subjects at steady state provides an average maximum naltrexone plasma concentration Cₘₐₓ of 0.8 to 2.3 ng/mL, such as 1.1 to 1.8 ng/mL. Exemplary naltrexone Cₘₐₓ values include those greater than or equal to 0.8, 0.9, 1.0, 1.1, 1.2 or 1.3 ng/ml and/or those less than or equal to 2.3, 2.2, 2.1, 2, 1.9, 1.8, 1.7, 1.6 and 1.5 ng/mL.

In one or more embodiments, administration of the oral dosage form comprising 32 mg of naltrexone a group of subjects at steady state provides an average naltrexone plasma area under the curve AUC₀₋₂₄ₕ of 10 to 28 ng*h/mL, such as 13 to 22 ng*h/mL. Exemplary naltrexone AUC₀₋₂₄ₕ values include those greater than or equal to 10, 11, 12, 13, 14, 15, 16 or 17 ng*h/mL and/or those less than or equal to 28, 27, 26, 25, 24, 23, 22, 21, 20, 19 or 18 ng*h/mL.

In one or more embodiments, administration of the oral dosage form comprising 360 mg of bupropion to a group of subjects at steady state provides an average maximum bupropion plasma concentration Cₘₐₓ of 120 to 330 ng/ml, such as 150 to 260 ng/mL. Exemplary bupropion Cₘₐₓ values include those greater than or equal to 120, 130, 140, 150, 160, 170, 180, 190 or 200 ng/ml and/or those less than or equal to 330, 320, 310, 300, 290, 280, 270, 260, 250, 240, 230, 220 or 210 ng/mL.

In one or more embodiments, administration of the oral dosage form comprising 360 mg of bupropion to a group of subjects at steady state provides an average bupropion plasma area under the curve AUC₀₋₂₄ₕ of 1,700 to 4,500 ng*h/mL, such as 2,200 to 3,600 ng*h/mL. Exemplary bupropion AUC₀₋₂₄ₕ values include those greater than or equal to 1,700, 1,800, 1,900, 2,000, 2,100, 2,200, 2,300, 2,400, 2,500, 2,600 or 2,700 ng*h/mL and/or those less than or equal to 4,500, 4,400, 4,300, 4,200, 4,100, 4,000, 3,900, 3,800, 3,700, 3,600, 3,500, 3,400, 3,300, 3,200, 3,100 or 3,000 ng*h/mL.

In one or more embodiments, the oral dosage form, when administered to a human subject once daily, is bioequivalent to a US FDA-approved trilayer tablet dosage form comprising naltrexone hydrochloride and bupropion hydrochloride (e.g. CONTRAVE®) administered to a human subject twice daily, under the bioequivalence parameters of: (a) a 90% Confidence Interval for AUC which is between 80% and 125%, and (b) a 90% Confidence Interval for Cₘₐₓ, which is between 80% and 125%.

In some embodiments, the administration of naltrexone and bupropion is continued for a period of, or of about, 1, 2, 3, 4, 6, 8, 10, 12, 16, 20, 24, 36, 48, or 52 weeks or more weeks, or a range defined by any two of the preceding values. In some embodiments, the administration of naltrexone and bupropion is continued until the reduction in symptoms of a disease, disorder, or condition is stabilized for a period of, or of about, 1, 2, 3, 4, 6, 8, 10, 12, 16, 20, 24, 36, 48, or 52 weeks or more weeks, or a range defined by any two of the preceding values. In some embodiments, administration of naltrexone and bupropion is continued until the individual no longer needs a treatment.

### EXAMPLES

### Example 1—Naltrexone-Containing Multilayer Extended-Release Beads

Exemplary formulations for naltrexone-containing multilayer extended-release beads are provided in Tables 1 and 2 below:

TABLE 1
Naltrexone HCl ER Beads

| Formulation | | | | | | | | |
|---|---|---|---|---|---|---|---|---|
| component | Formulation N1 | | Formulation N2 | | Formulation N3 | | Formulation N4 | |
| Total wt. gain % | 8.6 | | 10.8 | | 13 | | 8.8 | |
| (Based on | | | | | | | | |
| process | | | | | | | | |
| efficiency) | | | | | | | | |
| Plasticizer level | 10 | | 10 | | 10 | | 10 | |
| (% of dry | | | | | | | | |
| polymer) | | | | | | | | |
| Pore former level | 25 | | 25 | | 25 | | 20 | |
| (% of dry | | | | | | | | |
| polymer) | | | | | | | | |
| | mg/unit | % w/w | mg/unit | % w/w | mg/unit | % w/w | mg/unit | % w/w |
| Material | dose | (unit dose) | dose | (unit dose) | dose | (unit dose) | dose | (unit dose) |
| Naltrexone | 118.37 | 88.85 | 118.37 | 87.12 | 118.37 | 85.45 | 115.32 | 89.95 |
| Layered Beads | | | | | | | | |

Seal Coating*

| | | | | | | | | |
|---|---|---|---|---|---|---|---|---|
| Hydroxypropyl | 3.551 | 2.67 | 3.551 | 2.61 | 3.551 | 2.56 | 3.46 | 2.7 |
| cellulose (Klucel | | | | | | | | |
| EXF) | | | | | | | | |
| Talc 194 M | 0.71 | 0.53 | 0.71 | 0.52 | 0.71 | 0.51 | 0.692 | 0.54 |
| Dehydrated | 66.756 | N/A | 66.756 | N/A | 66.756 | N/A | 65.048 | N/A |
| ethanol\hat{ } | | | | | | | | |
| Total | 122.631 | 92.05 | 122.631 | 90.25 | 122.631 | 88.52 | 118.78 | 93.19 |

ER coating\hat{ }\hat{ }

| | | | | | | | | |
|---|---|---|---|---|---|---|---|---|
| Ethyl cellulose | 7.848 | 5.89 | 9.81 | 7.22 | 11.773 | 8.5 | 7.246 | 5.65 |
| 20 cps | | | | | | | | |
| Hypromellose 5 | 1.962 | 1.47 | 2.453 | 1.81 | 2.943 | 2.13 | 1.449 | 1.13 |
| cps (Methocel | | | | | | | | |
| E5LV) | | | | | | | | |
| Triethyl citrate | 0.785 | 0.59 | 0.981 | 0.72 | 1.177 | 0.85 | 0.725 | 0.57 |
| Acetone\hat{ } | 104.537 | N/A | 130.674 | N/A | 156.811 | N/A | 92.944 | N/A |
| Dehydrated | 13.067 | N/A | 16.334 | N/A | 19.601 | N/A | 11.618 | N/A |
| ethanol\hat{ } | | | | | | | | |
| Purified water\hat{ } | 13.067 | N/A | 16.334 | N/A | 19.601 | N/A | 11.618 | N/A |
| Total | 133.226 | 100 | 135.875 | 100 | 138.524 | 100 | 128.2 | 100 |

\hat{ }Evaporates during processing,
*Solid content of Seal Coat Solution = 6.0% w/w,
\hat{ }\hat{ }Solid content of ER Coat Solution = 7.5% w/w, Acetone:Dehydrated ethanol:purified water = 80:10:10

TABLE 2
Naltrexone HCl ER Beads

| Formulation component | Formulation N5 | |
|---|---|---|
| Solvent use for drug layering | 100% dehydrated ethanol | |
| Drug substance to binder ratio | 90:10 | |
| Antioxidant used in drug layering solution | Ascorbic acid | |
| Antioxidant level | 2.9% of DS | |
| BHT level | 9.4% of DS | |
| Solid content of drug layering solution | 8% w/w | |
| Theoretical polymer wt gain % | 7.5 | |
| Theoretical total wt gain % | 10.1 | |
| Plasticizer level (% of dry polymer) | 10 | |
| Pore former level (% of dry polymer) | 25 | |
| | mg/unit | % w/w |
| Material | dose | (unit dose) |

Substrate

| | | |
|---|---|---|
| MCC spheres (Actillets 350) | 60 | 46.96 |

Seal Coating-I

| | | |
|---|---|---|
| Ethyl cellulose 20 cps | 7.1 | 5.56 |
| Triethyl citrate | 0.71 | 0.56 |
| Acetone\hat{ } | 77.06 | N/A |
| Dehydrated ethanol\hat{ } | 9.63 | N/A |
| Purified water\hat{ } | 9.63 | N/A |
| Total | 67.81 | 53.08 |

Drug layering

| | | |
|---|---|---|
| Naltrexone HCl anhydrous (Form L) | 32 | 25.05 |
| Hydroxypropyl cellulose (Klucel EXF) | 3.2 | 2.5 |
| Ascorbic acid | 0.92 | 0.72 |
| Butylated hydroxy toluene (BHT) | 3 | 2.35 |
| Dehydrated ethanol\hat{ } | 449.88 | N/A |
| Total | 106.93 | 83.7 |

Seal Coating-II

| | | |
|---|---|---|
| Hydroxypropyl cellulose (Klucel EXF) | 3.21 | 2.51 |
| Dehydrated ethanol\hat{ } | 36.89 | N/A |
| Total | 110.138 | 86.21 |

ER coating

| | | |
|---|---|---|
| Ethyl cellulose 20 cps | 10.297 | 8.06 |
| Hypromellose 5 cps (Methocel E5LV) | 2.574 | 2.02 |
| Triethyl citrate | 1.03 | 0.81 |
| Acetone\hat{ } | 131.157 | N/A |
| Dehydrated ethanol\hat{ } | 17.145 | N/A |
| Purified water\hat{ } | 17.145 | N/A |
| Total | 124.039 | 97.1 |

Film (Top) coating

| | | |
|---|---|---|
| Opadry TF white 266F180001 | 3.721 | 2.91 |
| Dehydrated ethanol\hat{ } | 70.699 | N/A |
| Total | 127.76 | 100 |

\hat{ }Evaporates during processing, Solid content of Seal Coat-I Solution = 7.5% w/w, Acetone:Dehydrated ethanol:purified water = 80:10:10, ¥Solid content of Drug layering dispersion = 8.0% w/w, Solid content of Seal Coat-II Solution = 6.0% w/w, Solid content of ER Coat Solution = 7.5% w/w, Acetone:Dehydrated ethanol:purified water = 80:10:10, Solid content of Film coat dispersion = 8.0% w/w,

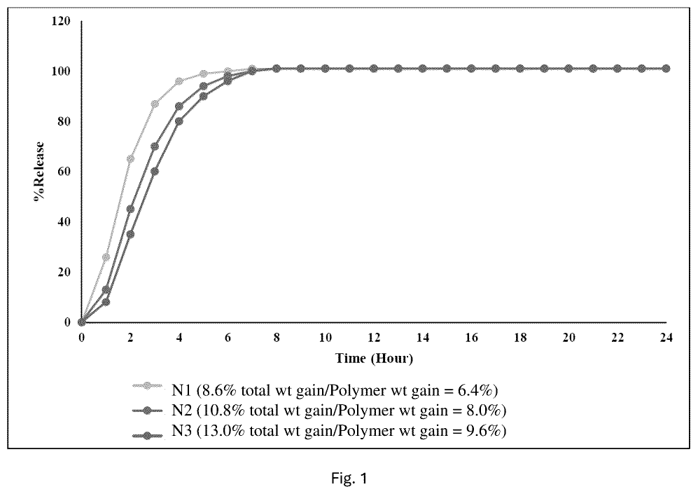

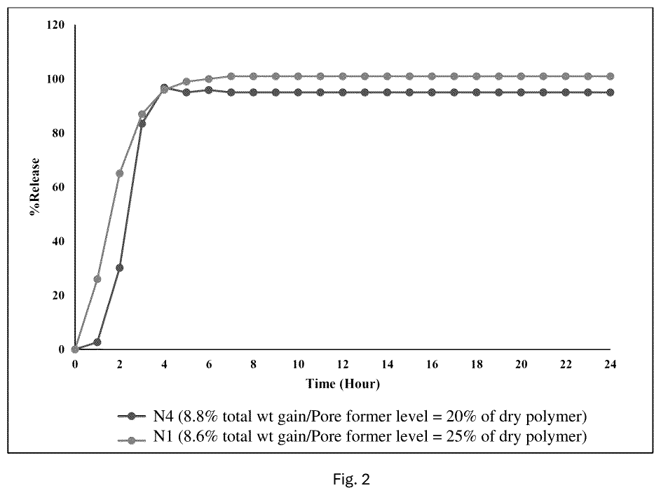

The in vitro dissolution naltrexone-containing multilayer beads of Formulations N1, N2, N3 and N4 were tested in USP Apparatus 1 Basket Method at 100 rpm in a dissolution medium of 900 mL purified water at 37° C. The in vitro release of naltrexone for Formulations N1, N2 and N3 is shown in FIG. 1 and Formulations N1 and N4 is shown in FIG. 2. As can be seen from FIG. 1, the release rate of the naltrexone HCl is inversely proportional to the coating thickness or polymer weight gain. As can be seen from FIG. 2, the release rate of the naltrexone HCl is increased by higher levels of pore former (20% pore former for N4 and 25% pore former for N1).

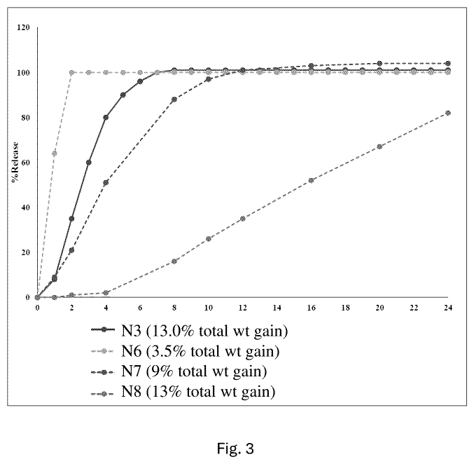

The in vitro dissolution naltrexone-containing multilayer beads of Formulation N5 with various levels of ER coating (N6=3.5% total wt gain, N7=9% total weight gain and N8=13% total weight gain) were tested in USP Apparatus 1 Basket Method at 100 rpm in a dissolution medium of 900 mL purified water at 37° C. The in vitro release of naltrexone for Formulations N6, N7 and N8 compared to N3 is shown in FIG. 3. Although N3 and N8 have similar weight gain, N8 has a slower release rate due to the increased homogeneity/sphericity of the naltrexone beads. Accordingly, the release rate for beads with a high level of homogeneity/sphericity can be increased by lowering the ER coating thickness/weight gain, increasing plasticizer levels and/or increasing pore former levels.

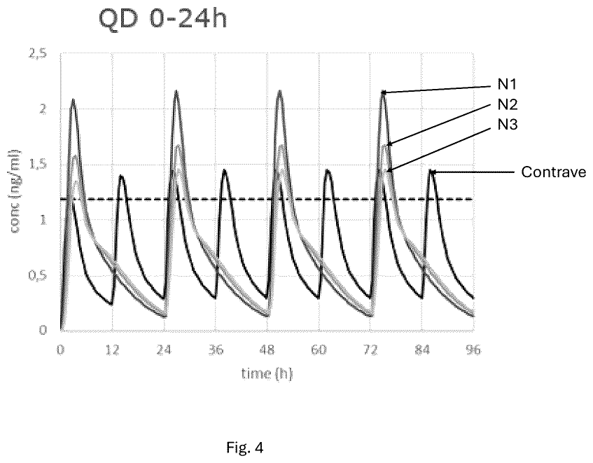

Based on in vitro release profiles, pharmacokinetic simulations for rate and extent of absorption at steady state (Cmax₍ₛₛ₎ and AUCₛₛ) were generated for selected prototypes of naltrexone HCl ER beads and compared with 16 mg BID (twice a day) dosing of CONTRAVE® tablets. The results are shown in FIG. 4 and Table 3 below:

TABLE 3
Pharmacokinetic (PK) parameters of prototypes of Naltrexone HCl ER beads
for 32 mg QD & CONTRAVE ® tablets 16 mg BID.

| | | | Total daily | | | | |
|---|---|---|---|---|---|---|---|
| | Dose of | | dose of | | | | |
| | Naltrexone | Dosing | Naltrexone | Cₘₐₓₛₛ | Cₘᵢₙₛₛ | AUCss | |
| Type | HCl (mg) | frequency | HCl (mg) | (ng/ml) | (ng/ml) | (ng * h/ml) | Fa (%) |
| CONTRAVE ® | 2*8 | BID | 32 | 1.44 | 0.29 | 17.4 | 83 |
| tablets | | | | | | | |
| N1 | 1*32 | QD | 32 | 2.16 | 0.13 | 16.7 | 80 |
| N2 | 1*32 | QD | 32 | 1.67 | 0.15 | 15.8 | 76 |
| N3 | 1*32 | QD | 32 | 1.45 | 0.16 | 15.4 | 74 |

*Simulations are generated using Gastroplus (G+) software, which removes all existing formulation in the GI tract when a new dose is given

Based on pharmacokinetic parameters shown in FIG. 4 and Table 3, formulations N2 and N3 are simulated to fall within bioequivalence criteria of 80%-120% for Cmax₍ₛₛ₎ and AUCₛₛ when comparing 32 mg QD (once a day) dosing of formulations N2 and N3 with 16 mg BID (twice a day) dosing of CONTRAVE® tablets.

### Example 2—Bupropion-Containing Multilayer Extended-Release Beads

Exemplary formulations for bupropion-containing multilayer extended-release beads are provided in Tables 4, 5 and 6 below:

TABLE 4
Bupropion HCl ER Beads

| Formulation | | | | | | | | |
|---|---|---|---|---|---|---|---|---|
| component | Formulation B1 | | Formulation B2 | | Formulation B3 | | Formulation B4 | |
| Drug loading in core | 79.6 | | 79.6 | | 79.6 | | 79.6 | |
| beads (% w/w) | | | | | | | | |
| Practical polymer wt. | 4.3 | | 5.1 | | 6 | | 7.7 | |
| gain (%) (Based on | | | | | | | | |
| process efficiency) | | | | | | | | |
| Total wt. gain % | 6.8 | | 8.1 | | 9.5 | | 12.2 | |
| (Based on process | | | | | | | | |
| efficiency) | | | | | | | | |
| Plasticizer level (% of | 21.3 | | 21.3 | | 21.3 | | 21.3 | |
| dry polymer) | | | | | | | | |
| Pore former level | 25 | | 25 | | 25 | | 25 | |
| (% of dry polymer) | | | | | | | | |
| Solvent System | Aqueous | | Aqueous | | Aqueous | | Aqueous | |
| | mg/ | % w/w | mg/ | % w/w | mg/ | % w/w | mg/ | % w/w |
| | unit | (unit | unit | (unit | unit | (unit | unit | (unit |
| Material | dose | dose) | dose | dose) | dose | dose) | dose | dose) |
| Bupropion Core | 452.25 | 90.89 | 452.25 | 89.82 | 452.25 | 88.66 | 452.25 | 86.53 |
| Beads | | | | | | | | |

Seal Coating*

| | | | | | | | | |
|---|---|---|---|---|---|---|---|---|
| Hypromellose 5 cps | 13.568 | 2.73 | 13.568 | 2.69 | 13.568 | 2.66 | 13.568 | 2.6 |
| (Methocel E5LV) | | | | | | | | |
| L-Cysteine | 0.136 | 0.03 | 0.136 | 0.03 | 0.136 | 0.03 | 0.136 | 0.03 |
| hydrochloride | | | | | | | | |
| monohydrate | | | | | | | | |
| Purified water\hat{ } | 214.696 | N/A | 214.696 | N/A | 214.696 | N/A | 214.696 | N/A |
| Total | 465.954 | 93.65 | 465.954 | 92.54 | 465.954 | 91.35 | 465.954 | 89.16 |

ER coating\hat{ }\hat{ }\hat{ }

| | | | | | | | | |
|---|---|---|---|---|---|---|---|---|
| Surelease ® E-7 19020 | 26.634 | 5.35 | 31.601 | 6.28 | 37.177 | 7.29 | 47.711 | 9.13 |
| EC Clear dispersion | | | | | | | | |
| Type-Bα | | | | | | | | |
| Hypromellose 5 cps | 5.009 | 1.01 | 5.941 | 1.18 | 6.989 | 1.37 | 8.97 | 1.72 |
| (Methocel E5LV) | | | | | | | | |
| Purified water\hat{ } | 20.099 | N/A | 23.845 | N/A | 28.053 | N/A | 36.002 | N/A |
| Total | 497.597 | 100 | 503.496 | 100 | 510.12 | 100 | 522.635 | 100 |

\hat{ }Evaporates during processing,
*Solid content of Seal Coat Solution = 6.0% w/w,
\hat{ }\hat{ }\hat{ }Solid content of ER Coat dispersion = 15% w/w,
**Surelease E-7 19020 dispersion contains 25% Solid content (18.8% of Ethyl cellulose 20 cps, 4% of Dibutyl sebacate, 2.2% of Oleic acid) and 75% liquid content (70.6% of purified water & 4.4% of Ammonium hydroxide 28%).

TABLE 5
Bupropion HCl ER Beads

| Formulation component | Formulation B5 | | Formulation B6 | | Formulation B7 | |
|---|---|---|---|---|---|---|
| Drug loading in core | 70 | | 70 | | 70 | |
| beads (% w/w) | | | | | | |
| Practical polymer wt. | 6 | | 9.5 | | 13 | |
| gain (%) (Based on | | | | | | |
| process efficiency) | | | | | | |
| Total wt. gain % (Based | 8.1 | | 12.8 | | 17.6 | |
| on process efficiency) | | | | | | |
| Plasticizer level (% of dry | 10 | | 10 | | 10 | |
| polymer) | | | | | | |
| Pore former level (% of | 25 | | 25 | | 25 | |
| dry polymer) | | | | | | |
| Solvent System | Organic | | Organic | | Organic | |
| | | % w/w | | % w/w | | % w/w |
| | mg/unit | (unit | mg/unit | (unit | mg/unit | (unit |
| Material | dose | dose) | dose | dose) | dose | dose) |
| Bupropion Core Beads | 514.32 | 89.79 | 514.32 | 86.03 | 514.32 | 82.57 |

Seal Coating*

| | | | | | | |
|---|---|---|---|---|---|---|
| Hypromellose 5 cps | 15.43 | 2.69 | 15.43 | 2.58 | 15.43 | 2.48 |
| (Methocel E5LV) | | | | | | |
| L-Cysteine hydrochloride | 0.154 | 0.03 | 0.154 | 0.03 | 0.154 | 0.03 |
| monohydrate | | | | | | |
| Purified water\hat{ } | 244.149 | N/A | 244.149 | N/A | 244.149 | N/A |
| Total | 529.904 | 92.51 | 529.904 | 88.64 | 529.904 | 85.08 |

ER coating\hat{ }\hat{ }\hat{ }

| | | | | | | |
|---|---|---|---|---|---|---|
| Ethyl cellulose 20 cps | 31.794 | 5.55 | 50.34 | 8.42 | 68.888 | 11.06 |
| Hypromellose 5 cps | 7.949 | 1.39 | 12.585 | 2.11 | 17.222 | 2.77 |
| (Methocel E5LV) | | | | | | |
| Dibutyl sebacate | 3.18 | 0.56 | 5.034 | 0.84 | 6.889 | 1.11 |
| Acetone\hat{ } | 423.507 | N/A | 670.529 | N/A | 920.07 | N/A |
| Dehydrated ethanol\hat{ } | 52.938 | N/A | 83.816 | N/A | 115.09 | N/A |
| Purified water\hat{ } | 52.938 | N/A | 83.816 | N/A | 115.09 | N/A |
| Total | 497.597 | 100 | 597.863 | 100 | 622.903 | 100 |

\hat{ }Evaporates during processing,
*Solid content of Seal Coat Solution = 6.0% w/w,
\hat{ }\hat{ }\hat{ }Solid content of ER Coat dispersion = 7.5%, Acetone:Dehydrated ethanol:purified water = 80:10:10.

TABLE 6
Bupropion HCl ER Beads

| Formulation component | Formulation B8 | | Formulation B9 | | Formulation B10 | |
|---|---|---|---|---|---|---|
| Drug loading in core beads | 75 | | 75 | | 70 | |
| (% w/w) | | | | | | |
| Practical polymer wt. gain | 7 | | 13 | | 4.8 | |
| (%) (Based on process | | | | | | |
| efficiency) | | | | | | |
| Total wt. gain % (Based on | 11.1 | | 20.5 | | 6 | |
| process efficiency) | | | | | | |
| Plasticizer level (% of dry | 21.3 | | 21.3 | | 10 | |
| polymer) | | | | | | |
| Pore former level (% of | 25 | | 25 | | 15 | |
| dry polymer) | | | | | | |
| Solvent System | Aqueous | | Aqueous | | Organic | |
| | | % w/w | | % w/w | | % w/w |
| | mg/unit | (unit | mg/unit | (unit | mg/unit | (unit |
| Material | dose | dose) | dose | dose) | dose | dose) |
| Bupropion Core Beads | 480 | 86.94 | 480 | 80.52 | 514.32 | 91.57 |

Seal Coating*

| | | | | | | |
|---|---|---|---|---|---|---|
| Hypromellose 5 cps | 14.4 | 2.61 | 14.4 | 2.42 | 15.43 | 2.75 |
| (Methocel E5LV) | | | | | | |
| L-Cysteine hydrochloride | 0.144 | 0.03 | 0.144 | 0.02 | 0.154 | 0.03 |
| monohydrate | | | | | | |
| Purified water\hat{ } | 227.856 | N/A | 227.856 | N/A | 244.149 | N/A |
| Total | 494.544 | 89.58 | 494.544 | 82.96 | 529.04 | 94.35 |

ER coating\hat{ }\hat{ }\hat{ }

| | | | | | | |
|---|---|---|---|---|---|---|
| Surelease ® E-7 19020 EC | 46.035 | 8.34 | 85.493 | 14.34 | N/A | N/A |
| Clear dispersion Type-Bα | | | | | | |
| Ethyl cellulose 20 cps | N/A | N/A | N/A | N/A | 25.435 | 4.53 |
| Hypromellose 5 cps | 11.509 | 2.08 | 16.073 | 2.7 | 3.815 | 0.68 |
| (Methocel E5LV) | | | | | | |
| Dibutyl sebacate | N/A | N/A | N/A | N/A | 2.544 | 0.45 |
| Acetone\hat{ } | N/A | N/A | N/A | N/A | 351.345 | N/A |
| Dehydrated ethanol\hat{ } | N/A | N/A | N/A | N/A | 43.918 | N/A |
| Purified water\hat{ } | 122.47 | N/A | 226.202 | N/A | 43.918 | N/A |
| Total | 552.088 | 100 | 596.11 | 100 | 561.698 | 100 |

\hat{ }Evaporates during processing,
*Solid content of Seal Coat Solution = 6.0% w/w,
\hat{ }\hat{ }\hat{ }Solid content of ER Coat dispersion = 15% w/w for B8 and B9 and 7.5% w/w for B10,
**Surelease E-7 19020 dispersion contains 25% Solid content (18.8% of Ethyl cellulose 20 cps, 4% of Dibutyl sebacate, 2.2% of Oleic acid) and 75% liquid content (70.6% of purified water & 4.4% of Ammonium hydroxide 28%), Acetone:Dehydrated ethanol:purified water for B10 = 80:10:10.

TABLE 7
Bupropion HCl ER Beads

| Formulation component | Formulation B11 | |
|---|---|---|
| Drug loading in core beads (% w/w) | 82 | |
| Theoretical polymer wt gain % | 6 | |
| Theoretical total wt gain % | 8.1 | |
| Plasticizer level (% of dry polymer) | 10 | |
| Pore former level (% of dry polymer) | 25 | |
| | mg/unit | % w/w |
| Material | dose | (unit dose) |

Core beads

| | | |
|---|---|---|
| Bupropion hydrochloride (Micronized) | 360 | 72.47 |
| Microcrystalline cellulose (Avicel PH 101) | 59.95 | 12.07 |
| Methylcellulose (Methocel A15LV) | 12.49 | 2.51 |
| L-Cysteine hydrochloride monohydrate | 4.39 | 0.88 |
| Colloidal anhydrous silica (Aerosil ® 200) | 2.2 | 0.44 |
| Purified water\hat{ } | q.s. | q.s. |
| Total | 439.03 | 88.37 |

Seal Coating

| | | |
|---|---|---|
| Hypromellose 5 cps (Methocel E5LV) | 13.568 | 2.73 |
| L-Cysteine hydrochloride monohydrate | 0.136 | 0.03 |
| Purified water\hat{ } | 214.696 | N/A |
| Total | 452.734 | 91.13 |

ER coating

| | | |
|---|---|---|
| Ethyl cellulose 20 cps | 27.164 | 5.47 |
| Hypromellose 5 cps (Methocel E5LV) | 6.791 | 1.37 |
| Dibutyl sebacate | 2.716 | 0.55 |
| Acetone\hat{ } | 361.82 | N/A |
| Dehydrated ethanol\hat{ } | 45.23 | N/A |
| Purified water\hat{ } | 45.23 | N/A |
| Total | 489.405 | 98.52 |

Film (Top) coating

| | | |
|---|---|---|
| Opadry TF 271ZA100000 purple | 7.341 | 1.48 |
| Dehydrated ethanol\hat{ } | 66.069 | N/A |
| Total | 496.746 | 100 |

\hat{ }Evaporates during processing, Solid content of Seal Coat Solution = 6.0% w/w, \hat{ }\hat{ } Solid content of ER Coat dispersion = 7.5% w/w, Solid content of Film Coat dispersion = 10% w/w

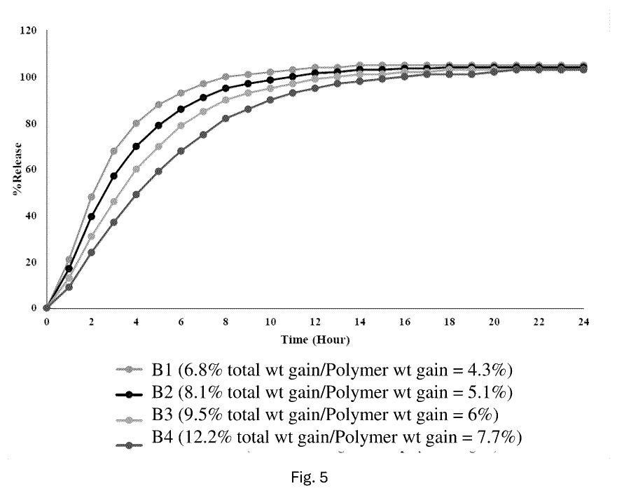

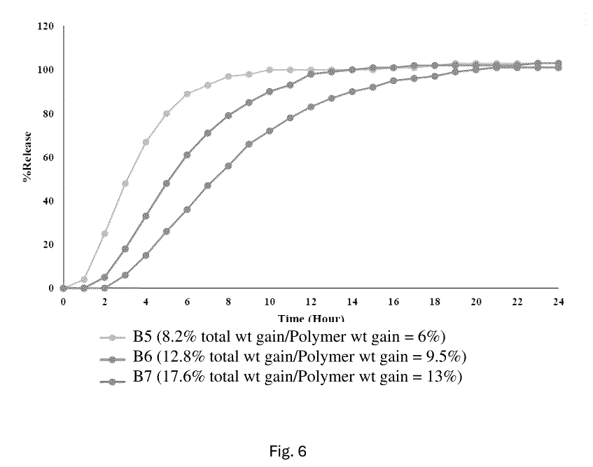

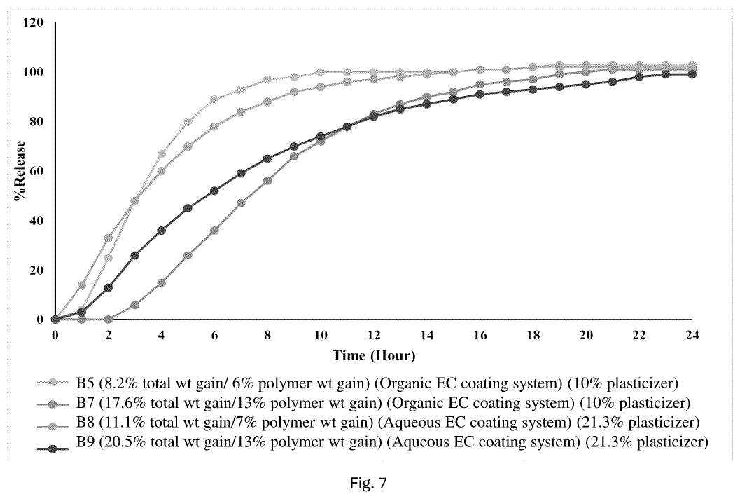

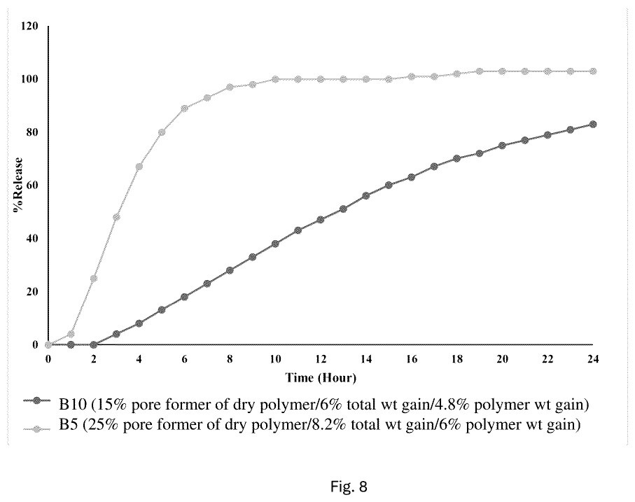

The in vitro dissolution of the bupropion-containing multilayer beads of Formulations B1, B2, B3, B4, B5, B6, B7, B8, B9 and B10 were tested in USP Apparatus 1 Basket Method at 100 rpm in a dissolution medium of 900 mL purified water at 37° C. The in vitro release of bupropion of Formulations B1, B2, B3 and B4 is shown in FIG. 5. As can be seen from FIG. 5, the release rate of the bupropion HCl is inversely proportional to the coating thickness or polymer weight gain. The in vitro release of bupropion Formulations B5, B6 and B7 is shown in FIG. 6. Again, as can be seen from FIG. 6, the release rate of the bupropion HCl is inversely proportional to the coating thickness or polymer weight gain. The in vitro release of bupropion Formulations B5, B7, B8 and B9 is shown in FIG. 7. As can be seen from FIG. 7, the release rate of the bupropion HCl is increased by higher levels of plasticizer (21.1% vs 10%). The in vitro release of bupropion Formulations B5 and B10 is shown in FIG. 8. As can be seen from FIG. 8, the release rate of the bupropion HCl is increased by higher levels of pore former (25% pore former for B5 and 15% pore former for B10).

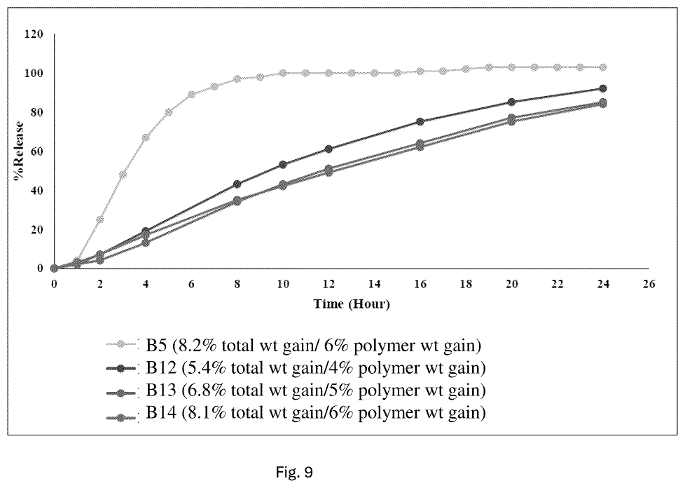

The in vitro dissolution bupropion-containing multilayer beads of Formulation B11 with various levels of ER coating (B12=5.4% total wt gain, B13=6.8% total weight gain and B14=8.1% total weight gain) were tested in USP Apparatus 1 Basket Method at 100 rpm in a dissolution medium of 900 mL purified water at 37° C. The in vitro release of bupropion for Formulations B12, B13 and B14 compared to B5 shown in FIG. 9. Although B5 and B14 have similar weight gain, B14 has a slower release rate due to the increased homogeneity/sphericity of the bupropion beads. Accordingly, the release rate for beads with a high level of homogeneity/sphericity can be increased by lowering the ER coating thickness/weight gain, increasing plasticizer levels and/or increasing pore former levels.

Based on in vitro release profiles, pharmacokinetic simulations for rate and extent of absorption at steady state (Cmax₍ₛₛ₎ and AUCₛₛ) were generated for selected prototypes of bupropion HCl ER beads and compared with 360 mg BID (twice a day) dosing of CONTRAVE® tablets. The results are shown in Table 8 below:

TABLE 8
Pharmacokinetic (PK) parameters of prototypes of Bupropion HCl ER beads
for 360 mg QD & CONTRAVE ® tablets 32 mg BID.

| | | | Total | | | | |
|---|---|---|---|---|---|---|---|
| | | | daily dose | | | | |
| | Dose of | | of | | | | |
| | Bupropion | Dosing | Bupropion | Cₘₐₓₛₛ | Cₘᵢₙₛₛ | AUCₛₛ | |
| Type | HCl (mg) | frequency | HCl (mg) | (ng/ml) | (ng/ml) | (ng * h/ml) | Fa (%) |
| Contrave ® | 2*90 | BID | 360 | 200 | 71 | 2852 | 96 |
| tablets | | | | | | | |
| B1 | 1*360 | QD | 360 | 258 | 42 | 2777 | 93 |
| B2 | 1*360 | QD | 360 | 220 | 44 | 2714 | 91 |
| B3 | 1*360 | QD | 360 | 187 | 46 | 2641 | 89 |
| B5 | 1*360 | QD | 360 | 202 | 45 | 2693 | 90 |
| B6 | 1*360 | QD | 360 | 152 | 47 | 2467 | 83 |
| B7 | 1*360 | QD | 360 | 135 | 47 | 2219 | 74 |

*Simulations are generated using Gastroplus (G+) software, which removes all existing formulation in the GI tract when a new dose is given

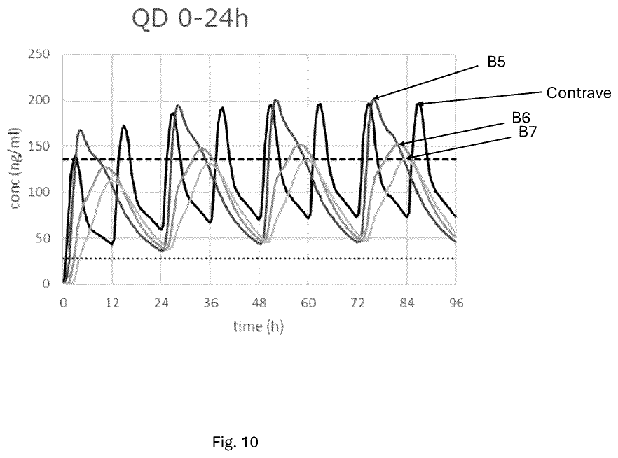

Based on pharmacokinetic parameters shown in FIG. 10 and Table 8, formulations B2, B3 and B5 are simulated to fall within bioequivalence criteria of 80%-120% for Cmax₍ₛₛ₎ and AUCₛₛ when comparing 360 mg QD (once a day) dosing of formulations B2, B3 and B5 16 mg BID (twice a day) dosing of CONTRAVE® tablets.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

1. An oral dosage form comprising a plurality of naltrexone-containing beads and a plurality of bupropion-containing beads,
wherein the naltrexone-containing beads are extended-release, multilayer beads comprising:
a core particle, wherein the core particle is drug-free;
an optional seal coating coated on the surface of the core particle;
a drug layer comprising naltrexone, or a salt thereof, coated on the surface of the core particle or, when present, the seal coating;
a non-aqueous barrier coating coated on the surface of the drug layer;
an extended-release coating coated on the surface of the barrier coating; and
an optional top coating coated on the surface of the extended-release coating;
wherein the naltrexone-containing beads have an in vitro naltrexone dissolution profile in a dissolution test of USP Apparatus 1 Basket Method at 100 rpm in a dissolution medium of water at 37° C. of:
i. 2% to 20% of the naltrexone released in one hour;
ii. 10% to 50% of the naltrexone released in two hours; and
iii. 50% to 90% of the naltrexone released in four hours; and
wherein the bupropion-containing beads are extended-release, multilayer beads comprising:
a core particle comprising bupropion, or a salt thereof;
an optional seal coating coated on the surface of the core particle;
an extended-release coating coated on the surface of the core particle, or if present, on the surface of the seal coating; and
an optional top coating coated on the surface of the extended-release coating.

2. The oral dosage form of claim 1, wherein the core particle of the naltrexone-containing beads is selected from a microcrystalline cellulose particle, a silica particle, and a sugar particle.

3. The oral dosage form of claim 1, wherein the drug layer of the naltrexone-containing beads comprises about 50% to about 95% by weight of naltrexone, or a salt thereof.

4. The oral dosage form of claim 3, wherein the drug layer of the naltrexone-containing beads comprises further comprises a low viscosity binder selected from Hypromellose 3 cps, Hypromellose 5 cps, and Hydroxypropyl cellulose.

5. The oral dosage form of claim 3, wherein the naltrexone comprises an anhydrous polymorph of the hydrochloride salt (Form L) and/or an ethanol solvate of the hydrochloride salt (Form F).

6. The oral dosage form of claim 5, wherein the non-aqueous barrier coating of the naltrexone-containing beads is prepared under anhydrous conditions.

7. The oral dosage form of claim 6, wherein the non-aqueous barrier coating of the naltrexone-containing beads is prepared using a solvent selected from the group consisting of class 3 solvents.

8. The oral dosage form of claim 1, wherein the naltrexone-containing beads comprise no more than 0.5% 2-chloro-10α-hydroxynaltrexone, based on the weight of naltrexone.

9. The oral dosage form of claim 1, wherein the bupropion, or a salt thereof, is present in the core particle of bupropion-containing beads ≥70% w/w.

10. The oral dosage form of claim 1, wherein the bupropion-containing beads comprise no more than 1% (2R,3R,5R)-2-(3-chlorophenyl)-2-hydroxy-3-methylthiomorpholine-5-carboxylic acid (RRR-CHMTCA) and no more than 0.5% (2S,3S,5R)-2-(3-chlorophenyl)-2-hydroxy-3-methylthiomorpholine-5-carboxylic acid (SSR-CHMTCA), based on the weight of bupropion.

11. The oral dosage form of claim 1, wherein the extended-release coating of the naltrexone-containing beads and/or the bupropion-containing beads comprises a release controlling polymer and a pore former.

12. The oral dosage form of claim 11, wherein the release controlling polymer is present in an amount of about 5% to about 25% by weight of the naltrexone-containing beads and/or the bupropion-containing beads.

13. The oral dosage form of claim 12, wherein the extended-release coating of the naltrexone-containing beads and/or the bupropion-containing beads comprises a plasticizer present in an amount of about 5% to about 15% of dry polymer concentration.

14. The oral dosage form of claim 1, wherein the naltrexone-containing beads comprise about 8 to about 32 mg of naltrexone or a salt thereof; and
wherein the bupropion-containing beads comprise about 90 mg to about 360 mg of bupropion or a salt thereof.

15. The oral dosage form of claim 14, wherein the in vitro naltrexone dissolution profile in the dissolution test is 30% to 80% of the naltrexone released in three hours.

16. The oral dosage form of claim 15, wherein the bupropion-containing beads have an in vitro bupropion dissolution profile in a dissolution test of USP Apparatus 1 Basket Method at 100 rpm in a dissolution medium of water at 37° C. of:
i. less than 30% of the bupropion released in one hour; or
ii. less than 60% of the bupropion released in two hours.

17. The oral dosage form of claim 16, wherein the in vitro bupropion dissolution profile in the dissolution test is:
i. 2% to 20% of the bupropion released in one hour; and
ii. 10% to 50% of the bupropion released in two hours.

18. The oral dosage form of claim 14, wherein the in vitro naltrexone dissolution profile in the dissolution test is 30% to 80% of the naltrexone released in three hours;
wherein the bupropion-containing beads have an in vitro bupropion dissolution profile in a dissolution test of USP Apparatus 1 Basket Method at 100 rpm in a dissolution medium of water at 37° C. of:
i. 2% to 20% of the bupropion released in one hour;
ii. 10% to 50% of the bupropion released in two hours; and
iii. 50% to 90% of the bupropion released in four hours.

19. The oral dosage form of claim 14, wherein administration of the oral dosage form to a group of subjects at steady state provides an average maximum naltrexone plasma concentration Cₘₐₓ of 1.1 to 1.8 ng/mL.

20. The oral dosage form of claim 14, wherein administration of the oral dosage form to a group of subjects at steady state provides an average naltrexone plasma area under the curve AUC₀₋₂₄ₕ of 13 to 22 ng*h/mL.

21. The oral dosage form of claim 14, wherein administration of the oral dosage form to a group of subjects at steady state provides an average maximum bupropion plasma concentration Cₘₐₓ of 150 to 260 ng/mL.

22. The oral dosage form of claim 14, wherein administration of the oral dosage form to a group of subjects at steady state provides an average bupropion plasma area under the curve AUC₀₋₂₄ₕ of 2,200 to 3,600 ng*h/mL.

23. The oral dosage form of claim 14, wherein the oral dosage form, when administered to a human subject once daily, is bioequivalent to a US FDA-approved trilayer tablet dosage form comprising naltrexone hydrochloride and bupropion hydrochloride administered to a human subject twice daily, under the bioequivalence parameters of: (a) a 90% Confidence Interval for AUC which is between 80% and 125%, and (b) a 90% Confidence Interval for Cmax, which is between 80% and 125%.

24. The oral dosage form of claim 1, wherein the naltrexone salt is naltrexone hydrochloride.

25. The oral dosage form of claim 24, wherein the naltrexone hydrochloride is substantially free of amorphous naltrexone hydrochloride.

26. The oral dosage form of claim 1, wherein the oral dosage form comprises a capsule containing the plurality of naltrexone-containing beads and the plurality of bupropion-containing beads.

27. A method of treating overweight or obesity in a subject in need thereof, the method comprising administering to the subject the oral dosage form of claim 1.

28. A method of treating overweight or obesity in a subject in need thereof, the method comprising:
administering to the subject a first oral dosage form comprising about 8 mg of naltrexone or salt thereof and about 90 mg of bupropion or salt thereof, wherein the first oral dosage form is administered once a day for a first week;
administering to the subject a second oral dosage form comprising about 16 mg of naltrexone or salt thereof and about 180 mg of bupropion or salt thereof, wherein the second oral dosage form is administered once a day for a second week;
administering to the subject a third oral dosage form comprising about 24 mg of naltrexone or salt thereof and about 270 mg of bupropion or salt thereof, wherein the third oral dosage form is administered once a day for a third week; and
administering to the subject a fourth oral dosage form comprising about 32 mg of naltrexone or salt thereof and about 360 mg of bupropion or salt thereof, wherein the fourth oral dosage form is administered once a day for a fourth and subsequent weeks,
wherein each of the first oral dosage form, the second oral dosage form, the third oral dosage form and the fourth oral dosage form comprises a plurality of naltrexone-containing beads and a plurality of bupropion-containing beads, wherein the naltrexone-containing beads are extended-release, multilayer beads and wherein the bupropion-containing beads are extended-release, multilayer beads;
wherein the naltrexone-containing beads have an in vitro naltrexone dissolution profile in a dissolution test of USP Apparatus 1 Basket Method at 100 rpm in a dissolution medium of water at 37° C. of:
i. 2% to 20% of the naltrexone released in one hour;
ii. 10% to 50% of the naltrexone released in two hours; and
iii. 50% to 90% of the naltrexone released in four hours.

29. A method of treating overweight or obesity in a subject in need thereof, the method comprising administering to the subject an oral dosage form comprising naltrexone and bupropion once a day, wherein the oral dosage form comprises a naltrexone extended-release formulation having an in vitro naltrexone dissolution profile in a dissolution test of USP Apparatus 1 Basket Method at 100 rpm in a dissolution medium of water at 37° C. of:
iv. 2% to 20% of the naltrexone released in one hour;
V. 10% to 50% of the naltrexone released in two hours; and
vi. 50% to 90% of the naltrexone released in four hours.

30. The method of claim 29, wherein the oral dosage form is a capsule.
